(19) United States
(12) Patent Application Publication
(10) Pub. No.: US 2026/0325410 A1
(43) Pub. Date: Oct. 22, 2026

(54) A PROCESS FOR PRODUCING A PERSONAL HYGIENE PRODUCT IN THE FORM OF A SHEET AND THE PRODUCT THEREOF

(71) Applicant: CAPELLO LIMITED, Bexhill-On-Sea Sussex (GB)

(72) Inventors: Huimin TANG, Guangzhou (CN); Qiong GUO, Guangzhou (CN)

(21) Appl. No.: 19/491,515

(22) PCT Filed: Jun. 10, 2024
(86) PCT No.: PCT/EP2024/065991

§ 371 Date: Dec. 9, 2025

(30) Foreign Application Priority Data
Jun. 9, 2023 (GB) 2308661.4

(51) Int. Cl. A61K 8/02; A61K 8/44; A61K 8/46; A61K 8/81; A61Q 19/00

(52) U.S. Cl. CPC A61K 8/0208; A61K 8/442; A61K 8/466; A61K 8/8129; A61Q 19/007

(57) ABSTRACT

The present invention relates to a process for producing a personal hygiene product, the personal hygiene product being formed into a sheet-like or film-like form. The product produced by the invention is intended but not restricted for use as a shampoo or hair conditioning product. Preferably, the sheet-like or film-like product is biodegradable. Further, the invention relates to the product of the above process. Furthermore, the invention relates to an assembly of the personal hygiene product and a dissolvable cover.

### FIELD OF THE INVENTION

The present invention relates to a process for producing a personal hygiene product, the personal hygiene product being formed into a sheet-like or film-like form. The product produced by the invention is intended but not restricted for use as a shampoo or hair conditioning product. Preferably, the sheet-like or film-like product is biodegradable. Further, the invention relates to the product of the above process. Furthermore, the invention relates to an assembly of the personal hygiene product and a dissolvable cover.

### BACKGROUND TO THE INVENTION

In recent times the ease of travelling to different places has increased. It is now much simpler to travel across the globe. However, there is still a need for alternative travel friendly products which are easy to store and carry in one's luggage without the risk of spilling or leakage. Such products would also be convenient for use in other situations, such on camping or caravanning trips or simply to carry to the gym or to sports matches or other situations outside the home. In addition, the limits placed on the carriage of liquids in airline carry-on luggage make carrying bottles of liquids difficult. A further problem with liquid hygiene products is that they are heavy to carry. One such range of problematic travel products include personal hygiene products like shampoo, hair conditioner, body wash, hand wash etc. It is cumbersome to carry such products in containers or even in the form of bars because there is always a risk of some fall out or distortion.

In addition to this, the packaging of such personal hygiene products is usually in the form of a non-degradable plastic which is a significant environmental pollutant. Hence, it is highly desirable to provide an environmentally sustainable alternative of these products as well as their packaging.

Keeping the above in mind, the present invention provides a travel-friendly form and packaging for personal hygiene products, preferably in the form of a sheet or a film. Moreover, the products as well as their packaging according to the present invention are completely dissolvable in water and biodegradable in nature. Therefore, there are huge environmental benefits associated with the present invention.

### SUMMARY OF THE INVENTION

In one aspect, the present invention provides a process for producing a personal hygiene product in the form of a sheet comprising the following steps:
a) providing the following components:
   (i) one or more high temperature resistant personal hygiene ingredients;
   (ii) a carrier ingredient enabling sheet formation;
   (iii) one or more solvents;
b) emulsifying these components to form a composition; and
c) compressing and drying the composition in order to form the composition into a sheet.

Suitably, one or more steps of the above process are carried out at a temperature of at least about 50° C. Preferably one or more steps of the above process are carried out at a temperature of at least about 60° C. Particularly preferably one or more steps of the above process are carried out at a temperature of at least about 80° C.

Suitably, step (b) may be carried out at a temperature of from about 50° C. to about 100° C. Preferably step (b) may be carried out at a temperature of from about 70° C. to about 95° C. Particularly preferably step (b) may be carried out at a temperature of from about 80° C. to about 90° C.

A method of the invention may further comprise, between steps (b) and (c), filtering the composition. This can remove any lumps of the components that may be present, and ensure that the composition has been fully and evenly mixed.

Suitably, step (c) is carried out at elevated temperature, such as at least about 80° C. Preferably step (c) is carried out at least about 100° C. Particularly preferably step (c) may be carried out at a temperature of at least about 110° C. Especially preferably step (c) may be carried out at a temperature of from about 100° C. to about 120° C. Alternatively or additionally, step (c) may be carried out or at reduced pressure, such as a pressure no greater than about 500 mbar. Preferably step (c) is carried out at a pressure no greater than about 300 mbar. Particularly preferably step (c) is carried out at a pressure no greater than about 200 mbar.

Suitably, component (iii) comprises water. Preferably, component (iii) comprises sterilized deionized water. The high boiling point of water means that the method can be carried out at elevated temperatures without significant solvent evaporation.

Advantageously, the components are high temperature resistant so that there is no loss or evaporation of the distinctive properties of these components while performing the process steps at a high temperature range of at least about 60° C. It will be understood that the term “high temperature resistant” is used to describe components which will not undergo degradation/decomposition or evaporation, or participate in unwanted side reactions, when exposed to elevated temperatures, for example at least 60° C., suitably at least about 80° C., preferably at least about 100° C.

Preferably, step (b) is carried out with continuous mixing and/or stirring.

Suitably, components (i)-(iii) are provided in a reaction vessel suitable for carrying out the emulsification step (b), for example an emulsifier pot.

Suitably, step (b) is carried out in an emulsifier pot with continuous stirring or mixing. Preferably step (b) is carried out with continuous stirring or mixing at a speed of from about 30 to about 80 rpm. Particularly preferably step (b) is carried out with continuous stirring or mixing at a speed of from about 40 to about 60 rpm.

Preferably, the emulsifier pot is prepared before use by sterilizing it for about 30 minutes by using pure water at about 100° C. followed by cooling down the temperature of the pot to about 60° C.

Suitably, step b) comprises adding the above components to an emulsifier pot, preferably prepared as described above, with continuous stirring such that a complete and even dissolution of each previous component occurs prior to the addition of the next component.

The composition formed by step (b) may be an emulsion. The composition formed by step (b) may take the form of a thick or viscous liquid or stew.

Suitably, step (c) of the above process comprises the steps of:
i. taking the composition formed by emulsification step b);
ii. compressing the composition one or more times between rollers;
iii. feeding the compressed composition to a sheet forming machine wherein the composition is dried at elevated temperature or under reduced pressure to form a sheet.

The drying of the composition may involve evaporation of solvent(s).

Steps (ii) and/or (iii) as described above may be carried out at a temperature of at least about 80° C. Preferably step (ii) and/or (iii) is carried out at least about 100° C. Particularly preferably step (ii) and/or (iii) may be carried out at a temperature of at least about 110° C. Especially preferably step (ii) and/or (iii) may be carried out at a temperature of from about 100° C. to about 120° C.

Step (c) may further comprise, between steps (ii) and (iii), the step of filtering the compressed composition.

Methods of the invention lead to the formation of a personal hygiene product in the form of a sheet which is stable, compact, and convenient to travel with. Suitably, when the personal hygiene product in the form of a sheet is brought into contact with water, it can be applied to the user's body or hair and used as normal. For example, a shampoo in the form of a sheet may be combined with water to form a lather when applied to the user's hair. This may be facilitated by the presence of a surfactant.

The above process may further comprise one or more of the following steps:
a. slicing the sheet formed by step (c) by using a sheet cutting machine;
b. packaging the sliced sheet in a dissolvable cover.

The method of the invention may further comprise, between steps (b) and (c), the step of adding one or more additional components to the composition. This could be done at the temperature at which step (b) has been carried out. Alternatively, for example if step (b) was carried out at an elevated temperature, the composition may be cooled (for example to room temperature) before addition of the one or more additional components. Desirably, the addition temperature can be selected based on the properties, for example the volatility, of the one or more additional components.

The personal hygiene ingredient may be a shampoo. The personal hygiene ingredient may be a conditioner. The personal hygiene ingredient may be selected from the group comprising surfactants, hair conditioning agents, antipruritics, smoothing agents, and combinations thereof.

The surfactants may be selected from sodium cocoyl isethionate, potassium cocoyl glycinate, sodium lauroyl glutamate, cocamidopropyl betaine, sodium cocoyl glycinate, and combinations thereof.

The hair conditioning agent may be selected from the group comprising fruit oils, hydrolysed silk, conditioning polymers, smoothing agents, and combinations thereof. A suitable fruit oil may be Olive (Olea europaea) oil. A suitable conditioning polymer may be JR-400.

The antipruritic may be selected from Calendula officinalis flower oil, Rosmarinus officinalis (Rosemary) leaf oil, and combinations thereof.

The personal hygiene product may further comprise a humectant. Suitably the personal hygiene product may comprise glycerine as a humectant.

The personal hygiene product may also comprise a skin conditioning agent. Suitably the personal hygiene product may comprise a skin conditioning agent selected from the group comprising hexanediol, ethylhexylglycerin, and combinations thereof.

The personal hygiene product may also comprise flavours, fragrances or preservatives. Suitably the personal hygiene product may comprise phenoxyethanol as a preservative.

Preferably, the carrier component enabling sheet formation is polyvinyl alcohol. Polyvinyl alcohol serves as a strong binder and provides physical strength to the sheet formed.

Suitably, the solvent (iii) is a solvent in which components (i) and (ii) are soluble. Preferably, solvent (iii) is water, quite preferably sterilized deionized water. The carrier component may be present in an amount of from about 5 wt % to about 50 wt % based on the total weight of the composition. Suitably, the carrier component may be present in an amount of from about 10 wt % to about 40 wt % based on the total weight of the composition. Preferably, the carrier component may be present in an amount of from about 15 wt % to about 30 wt % based on the total weight of the composition. Particularly preferably, polyvinyl alcohol is present as the carrier component in an amount of from about 15 wt % to about 30 wt % based on the total weight of the composition.

It will be understood that all percentages are provided in weight percentages based on the total weight of the composition provided by step (b), i.e. including any solvents present and any optional further components. It will be further understood that because step (c) comprises drying of the composition and is optionally carried out at elevated temperatures, evaporation of the solvent(s) is expected and therefore the weight percentages of components in a personal hygiene product made by a process of the invention is expected to differ from the percentage ranges described herein.

Suitably, the composition comprises from about 30 wt % to about 70 wt % solvent based on the total weight of the composition. Preferably, the composition comprises from about 40 wt % to about 60 wt % solvent based on the total weight of the composition. Particularly preferably the composition comprises from about 40 wt % to about 60 wt % water based on the total weight of the composition.

Suitably, the composition comprises from about 5 wt % to about 30 wt % of one or more surfactants based on the total weight of the composition. Preferably, the composition comprises from about 10 wt % to about 25 wt % of one or more surfactants based on the total weight of the composition. Particularly preferably, the composition comprises from about 15 wt % to about 22 wt % of one or more surfactants based on the total weight of the composition.

Suitably, the composition comprises sodium cocoyl isethionate as a surfactant in an amount of from about 5 wt % to about 20 wt % based on the total weight of the composition. Preferably the composition comprises sodium cocoyl isethionate as a surfactant in an amount of from about 5 wt % to about 20 wt % based on the total weight of the composition

Suitably, the composition comprises one or more hair conditioning agents in an amount of from about 0.5 wt % to about 5 wt % based on the total weight of the composition. Preferably, the composition comprises one or more hair conditioning agents in an amount of from about 1 wt % to about 4 wt % based on the total weight of the composition.

Suitably, the composition comprises one or more antipruritics in an amount of from about 0.5 wt % to about 5 wt % based on the total weight of the composition. Preferably, the composition comprises one or more antipruritics in an amount of from about 1 wt % to about 4 wt % based on the total weight of the composition.

Suitably, the composition comprises one or more skin conditioning agents in an amount of from about 0.1 wt % to about 1 wt % based on the total weight of the composition. Preferably, the composition comprises one or more skin conditioning agents in an amount of from about 0.3 wt % to about 0.7 wt % based on the total weight of the composition.

Suitably, the composition comprises one or more humectants in an amount of from about 1 wt % to about 10 wt % based on the total weight of the composition. Preferably, the composition comprises one or more humectants in an amount of from about 2 wt % to about 7 wt % based on the total weight of the composition.

Suitably, the composition comprises one or more preservatives in an amount of from about 0.05 wt % to about 1 wt % based on the total weight of the composition. Preferably, the composition comprises one or more preservatives in an amount of from about 0.1 wt % to about 0.5 wt % based on the total weight of the composition.

Suitably, the composition comprises one or more flavours or fragrances in an amount of from about 0.05 wt % to about 1 wt % based on the total weight of the composition. Preferably, the composition comprises one or more flavours or fragrances in an amount of from about 0.1 wt % to about 0.5 wt % based on the total weight of the composition.

Suitably, the composition has a pH in the range of from about 5 to about 8. Preferably, the composition has a pH in the range of from about 6 to about 7.

In yet another aspect, the present invention provides a sheet form of the personal hygiene product obtained through the above process. Suitably, the sheet form of the personal hygiene product obtained through the above process is a shampoo sheet.

Suitably, the sheet form of the personal hygiene product according to the invention has a thickness ranging from about 0.5 mm to about 2 mm per sheet. Preferably the sheet form of the personal hygiene product has a thickness of from about 0.75 mm to about 1.5 mm. Particularly preferably the sheet form of the personal hygiene product has a thickness of from about 1 mm to about 1.2 mm.

In yet another aspect, the present invention provides an assembly of the sheet form of the personal hygiene product with a dissolvable cover that encloses the product. Suitably, an assembly of a shampoo sheet with a dissolvable cover that encloses the shampoo sheet is provided. Preferably, the cover is fully dissolvable in water. Particularly preferably, the cover is fully dissolvable in water at room temperature, for example at a temperature of as low as 15° C.

The advantages offered by the present invention include ease of packaging and transportation of the sheet form of the personal hygiene product. Products prepared according to the invention are convenient for the users to carry and travel with as well as offering sustainable and environmentally friendly alternatives to existing technology.

### DESCRIPTION OF THE DRAWINGS

FIG. 1 shows a flowchart demonstrating a process according to the invention.

### DETAILED DESCRIPTION

In one embodiment, for example, a sheet shampoo is prepared according to a method of the invention. The composition comprises the following ingredients as listed in Table 1 below.

TABLE 1

| Component | Weight % | Function |
|---|---|---|
| Sodium cocoyl isethionate | 7 | Surfactant |
| Potassium cocoyl glycinate | 2 | Surfactant |
| Sodium lauroyl glutamate | 5 | Surfactant |
| Cocamidopropyl betaine | 5 | Surfactant |
| Olea europaea (olive) fruit | 2 | Hair conditioning agent |
| oil | | |
| Glycerin | 5 | Humectant |
| Calendula officinalis flower | 2 | Antipruritic |
| oil | | |
| Rosmarinus officinalis | 1 | Antipruritic |
| (Rosemary) leaf oil | | |
| Aroma | 0.2 | Flavours and Fragrances |
| Ethylhexylglycerin | 0.5 | Skin conditioning agent |
| Phenoxyethanol | 0.3 | Preservative |
| Polyvinyl alcohol | 20 | Carrier |
| Sterilized deionized water | 50 | Solvent |

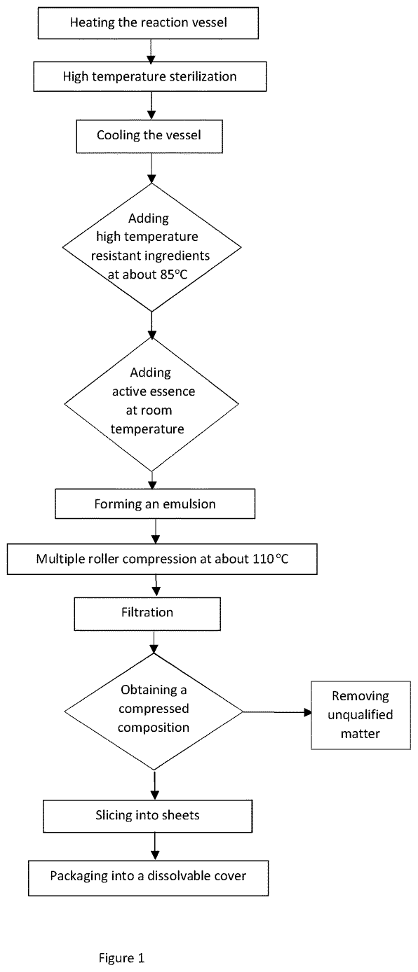

A method of the invention is shown in FIG. 1. Referring to the flow diagram in FIG. 1, an emulsifier pot is taken and subjected to sterilization by flowing heated water at about 100° C. through the pot. After carrying out the sterilization for about 30 minutes, the pot is subjected to a jacket cooling water system to bring down the temperature of the pot to about 60° C. Thereafter, at this temperature, the high temperature resistant components of the composition forming the personal hygiene product, including components such as surfactants, hair conditioning agents, antipruritics and carrier component are added in the emulsifier pot in a sequential manner, optionally in the presence of solvent, along with continuous stirring at around 50 rpm for a homogeneous mixing. Prior to the addition of a component, it is ensured that the previously added component has been homogenously dissolved in the solvent to avoid formation of lumps. Active essence such as flavours and fragrances are added in last after cooling the emulsifier pot to room temperature. This is because these active essence components otherwise lose their properties at high temperature. Following this, a thick composition thus formed is subjected to multiple roller compression and filtering to assume a further compact form. The compact form of the composition thus produced is fed into a sheet forming machine wherein it is dried at elevated temperature or under reduced pressure to from a sheet of the personal hygiene product. The sheet is then sliced using a sheet cutting machine and each slice is packaged in a dissolvable cover.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A process for producing a personal hygiene product in the form of a sheet, the process comprising the following steps:
a) providing the following components:
i) one or more high temperature resistant personal hygiene ingredients;
ii) a carrier ingredient enabling sheet formation;
iii) one or more solvents;
b) emulsifying the components to form a composition; and
c) compressing and drying the composition in order to form the composition into a sheet.

2. The process of claim 1, wherein step (c) comprises the steps of:
i. taking the composition formed by emulsification step b);
ii. compressing the composition one or more times between rollers;
i. feeding the compressed composition to a sheet forming machine wherein the composition is dried, optionally by evaporation of solvent(s) at elevated temperature or under reduced pressure, to form a sheet.

3. The process of claim 1, further comprising the step of filtering the composition formed by step (b).

4. The process of claim 1, wherein one or more steps are carried out at a temperature of at least about 50° C.

5. The process of claim 1, wherein step (b) is carried out at a temperature of at least about 50° C., preferably at a temperature of at least about 60° C.

6. The process of claim 1, wherein step (c) is carried out at a temperature of at least about 80° C., preferably at a temperature of at least about 100° C.

7. The process of claim 1, further comprising the step of adding one or more additional components to the composition between steps (b) and (c).

8. The process of claim 1, wherein step (b) is carried out with continuous mixing and/or stirring.

9. The process of claim 1, further comprising one or more of the following steps:
a) slicing the sheet formed by step (c) by using a sheet cutting machine;
b) packaging the sliced sheet in a dissolvable cover.

10. The process of claim 1, wherein the composition comprises one or more personal hygiene components selected from surfactants, hair conditioning agents, antipruritics, and a carrier component, optionally along with one or more solvents.

11. The process of claim 1, wherein the carrier component is polyvinyl alcohol.

12. The process of claim 1, wherein component (iii) comprises water.

13. The process of claim 1, wherein the composition comprises one or more surfactants selected from sodium cocoyl isethionate, potassium cocoyl glycinate, sodium lauroyl glutamate, cocamidopropyl betaine, sodium cocoyl glycinate, and combinations thereof.

14. The process of claim 1, wherein the composition comprises from about 5 wt % to about 30 wt % of one or more surfactants based on the total weight of the composition; and/or
wherein the carrier component is present in the composition in an amount of from about 5 wt % to about 50 wt % based on the total weight of the composition; and/or
wherein the one or more solvents are present in the composition in an amount of from about 30 wt % to about 70 wt % based on the total weight of the composition; and/or
wherein the composition comprises one or more hair conditioning agents in an amount of from about 0.5 wt % to about 5 wt % based upon the total weight of the composition; and/or
wherein the composition comprises one or more antipruritics in an amount of from about 0.5 wt % to about 5 wt % based upon the total weight of the composition;
and/or wherein the composition comprises one or more humectants in an amount of from about 1 wt % to about 10 wt % based upon the total weight of the composition.

15. (canceled)

16. (canceled)

17. (canceled)

18. (canceled)

19. (canceled)

20. The process of claim 1, wherein the composition further comprises one or more skin conditioning agents suitably in an amount of from about 0.1 wt % to about 1 wt % based upon the total weight of the composition.

21. The process of claim 1, wherein the composition further comprises one or more flavours, fragrances or preservatives.

22. The process of claim 1, wherein the composition comprises one or more preservatives in an amount of from about 0.05 wt % to about 1 wt % based upon the total weight of the composition.

23. A personal hygiene product, in the form of a sheet, made from the process of claim 1.

24. The personal hygiene product of claim 23, having a thickness of from about 0.5 mm to about 2 mm.

25. An assembly of the personal hygiene product as claimed in claim 23 with a dissolvable cover that encloses the said product.
